(19) United States
(12) Patent Application Publication
(10) Pub. No.: US 2026/0252868 A1
(43) Pub. Date: Aug. 27, 2026

(54) NEUROMORPHIC SYSTEM FOR MOLECULAR ELECTROMAGNETIC SIGNATURE DETECTION AND ADAPTIVE RFE MODULATION

(71) Applicant: NearField Atomics Inc., Anacortes, WA (US)

(72) Inventor: John T. Butters, Anacortes, WA (US)

(21) Appl. No.: 19/546,267

(22) Filed: Feb. 20, 2026

Related U.S. Application Data
(60) Provisional application No. 63/761,606, filed on Feb. 21, 2025.

(51) Int. Cl. G06N 3/063; G01R 33/09

(52) U.S. Cl. CPC G06N 3/063; G01R 33/091; G01R 33/098

(57) ABSTRACT

Identifying radio frequency electromagnetic (RFE) signal patterns or signatures of target molecules includes measuring an electromagnetic field of a target molecule and associating least one RFE signature with at least one function or structural feature of the target molecule. An effect of the target molecule on a biological sample is tested by applying an RFE signal on the sample according to an RFE signature and measuring changes in RFE output by the sample. A neuromorphic computing unit may extract a frequency‑energy feature vector and provide the vector to a generative model that synthesizes a radio frequency excitation signal that is delivered to the biological sample while a feedback controller adjusts the signal in real time to converge the signal emission toward producing a desired RFE output of the biological sample. The closed‑loop system operates with sub‑millisecond latency and sub‑watt power, enabling dynamic interrogation and modulation of biological or chemical processes.

### TECHNICAL FIELD

The disclosure relates to neuromorphic electronics, bioelectronic interfaces, and closed‑loop radio frequency (RF) systems. Aspects include measuring radio frequency electromagnetic (RFE) fields and applying radio frequency electromagnetic fields to substances.

### BACKGROUND

Ultra-low radio frequency energy of a substance, e.g., a target molecule, can be measured. Such measurement includes measuring a unique electrostatic potential of the target molecule which is a representation of structural and functional attributes of the target molecule.

Bioelectromagnetic signatures have been shown to correlate with metabolic and signaling states in biological tissue. Conventional spectrum‑analysis pipelines rely on graphics‑processing units (GPUs) executing Fourier transforms which require high power budgets, millisecond‑scale latency, and coarse frequency resolution.

### BRIEF SUMMARY

Aspects of the present disclosure concern a neuromorphic computing unit (NCU) that detects radio frequency electromagnetic (RFE) signals and, in real time, generates and delivers or causes to be generated and delivered targeted RF waveforms to a target substrate to modulate biological or chemical processes.

Application of specific ultra-low radio frequency energy may be used to induce electron and charge transfer in a defined target, for example a biological sample, producing altered cell dynamics in the sample that can be measured for a therapeutic response. In at least some embodiments, to provide therapy, an ultra-low radio frequency energy cognate of a target molecule is delivered locally and non-systemically via a medical device. To provide such therapy, an ultra-low radio frequency energy cognate of a target molecule to be applied is obtained.

Realtime modification or adjustment of the applied RFE signals by the NCU enables efficient and effective testing of target molecules that are represented by respective RFE signatures in a test environment.

The present disclosure includes for example the following enumerated Embodiments.

Embodiment 1. A neuromorphic computing system for molecular electromagnetic signature detection and adaptive radio frequency electromagnetic (RFE) modulation, comprising:

a radio frequency electromagnetic (RFE) application control and generation module configured to apply an RFE signal to a sample in a biological interface according to characteristics of an RFE signature;

a magnetoresistive (MR) sensor array arranged with respect to the biological interface to detect RFE signals from the sample;

a testing data acquisition (DAQ) module communicatively coupled to the MR sensor array to generate RFE signal data based on the detected RFE signals; and

a neuromorphic computing unit communicatively coupled to the testing DAQ module to receive the RFE signal data,

wherein the neuromorphic computing unit is configured to adjust in real time the RFE signal applied to the sample in the biological interface.

Embodiment 2. The neuromorphic computing system of Embodiment 1, wherein the RFE signal applied to the sample is generated according to the characteristics of an RFE signature selected from a library containing a plurality of RFE signatures.

Embodiment 3. The neuromorphic computing system of Embodiment 2, wherein the RFE signatures are obtained from measurement of RFE signals of solvated molecules and are representative of molecular structure and/or function of the solvated molecules.

Embodiment 4. The neuromorphic computing system of any one of Embodiments 1-3, wherein the neuromorphic computing unit is configured to determine a difference between the detected RFE signals and an RFE signal indicative of desired therapeutic benefit, and adjust in real time the RFE signal applied to the sample in the biological interface to minimize the difference.

Embodiment 5. The neuromorphic computing system of any one of Embodiments 1-4, wherein the magnetoresistive (MR) sensor array is comprised of tunnel magnetoresistance (TMR) sensors arranged to detect real-time changes in biological activity in the sample by measuring the RFE signals from the sample.

Embodiment 6. The neuromorphic computing system of any one of Embodiments 1-5, wherein the neuromorphic computing unit is configured to recognize one or more patterns in the RFE signal data and generate an RFE signature representative of the sample based on a pattern in the RFE signal data.

Embodiment 7. The neuromorphic computing system of Embodiment 6, wherein the neuromorphic computing unit is configured to compare the generated RFE signature with one or more RFE signatures stored in a library containing a plurality of RFE signatures.

Embodiment 8. The neuromorphic computing system of Embodiment 6 or Embodiment 7, wherein the neuromorphic computing unit uses spike-based, event-driven processes to recognize the one or more patterns in the RFE signal data.

Embodiment 9. The neuromorphic computing system of any one of Embodiments 1-8, wherein the neuromorphic computing unit is configured to correlate the RFE signal applied to the sample by the RFE application control and generation module with a biological response observed the detected RFE signals and assess an effectiveness of the RFE signal in eliciting a desired biological response in the sample.

Embodiment 10. The neuromorphic computing system of Embodiment 9, wherein the effectiveness is assessed by comparing a real-time measured biological response represented in the detected RFE signal to an expected, anticipated, or desired biological response, and a difference between the measured biological response and the expected, anticipated, or desired response triggers the real-time adjustment in the RFE signal applied to the sample.

Embodiment 11. A closed‑loop radio‑frequency excitation system, comprising:

a plurality of magnetic field sensors arranged to detect a radio frequency electromagnetic (RFE) signal from a sample;

a neuromorphic computing unit (NCU) configured to extract a frequency‑energy feature vector from the RFE signal detected by the plurality of magnetic field sensors;

an RFE signal generator configured to synthesize a radio frequency excitation signal responsive to the frequency‑energy feature vector;

an emitter assembly configured to deliver the radio frequency excitation signal to the target; and

a radio frequency electromagnetic (RFE) application control and generation module coupled to the magnetic field sensors and the RFE signal generator and operable to adjust the radio frequency excitation signal to reduce a difference between the extracted frequency‑energy feature vector and a threshold frequency‑energy feature vector.

Embodiment 12. The radio‑frequency excitation system of Embodiment 11, wherein the NCU comprises a spiking neural network core.

Embodiment 13. The radio‑frequency excitation system of Embodiment 11 or Embodiment 12, wherein the RFE signal generator comprises a variational autoencoder coupled to a generative adversarial network.

Embodiment 14. The radio‑frequency excitation system of any one of Embodiments 11-13, whereinthe threshold frequency‑energy feature vector is representative of a structural RFE motif that is linked to a desired therapeutic effect in the sample.

Embodiment 15. The radio‑frequency excitation system of any one of Embodiments 11-14, whereinthe NCU is configured to correlate a biological response of the sample with the extracted frequency‑energy feature vector and arrange the threshold feature-energy vector to represent a desired biological response of the sample.

Embodiment 16. The radio‑frequency excitation system of any one of Embodiments 11-15, wherein the RFE signal generator is configured to adjust one or more parameters of the radio frequency excitation signal to maintain the extracted frequency‑energy feature vector within a specified range of the threshold frequency‑energy feature vector.

Embodiment 17. A neuromorphic radio frequency electromagnetic system, comprising:

a plurality of magnetic field sensors arranged to detect a radio frequency electromagnetic (RFE) signal from a target;

a neuromorphic computing unit (NCU) configured to extract a frequency‑energy feature vector from the RFE signal detected by the magnetic field sensors and to generate a new RFE signal based on an observed RFE outcome or an experimental objective.

Embodiment 18. The system of Embodiment 17, further comprising an RFE molecular library communicatively coupled to the NCU, the NCU being configured to retrieve one or more RFE signals from the RFE molecular library to generate a new RFE signal based on an observed RFE outcome or an experimental objective.

Embodiment 19. The system of Embodiment 17 or Embodiment 18, further comprising a waveform generator configured to synthesize a radio frequency excitation signal responsive to the frequency‑energy feature vector.

Embodiment 20. The system of any one of Embodiments 17-19, wherein the NCU is configured to computationally generate a molecular candidate having an RFE signature corresponding to the new RFE signal, wherein the new RFE signal encodes a predicted structural or functional molecular attribute of the molecular candidate.

Embodiment 21. The system of any one of Embodiments 1-20, wherein the neuromorphic computing unit is implemented in software executed on one or more general-purpose processor.

Embodiment 22. The system of Embodiment 21, wherein the neuromorphic computing unit is implemented in software executed without specialized neuromorphic hardware.

Embodiment 23. A method of using the system of any one of Embodiments 1-22 comprising or causing to be performed or executed the function(s) described therein, optionally comprising one or more function(s) or the method recursively, further optionally with adjustment based on user input (e.g., based on an assessment of an initial performed function or cycle, and/or based on a desired outcome) following one or more performed function(s) or cycle(s) of the method.

### BRIEF DESCRIPTION OF THE DRAWINGS

For a better understanding of example embodiments of the present disclosure, reference will be made to the following Detailed Description, which is to be read in association with the accompanying drawings, wherein:

FIG. 1 is a diagram of one example of a magnetoresistive (MR) sensor;

FIG. 2 illustrates an arrangement of MR sensors in a Wheatstone bridge;

FIG. 3 is a schematic diagram of an arrangement of MR sensor devices around a container of solvated target molecule, according to the present disclosure;

FIG. 4 is a schematic diagram of another arrangement of MR sensor devices around a container of solvated target molecule, according to the present disclosure;

FIG. 5 is a schematic diagram of yet another arrangement of MR sensor devices around a container of solvated target molecule, according to the present disclosure;

FIG. 6 is a schematic diagram of the arrangement of MR sensor devices around a container of solvated target molecule of FIG. 3 inside a shield, according to the present disclosure;

FIG. 7 depicts a workflow for generating and employing ultra-low radio frequency energy using MR sensor devices, according to the present disclosure;

FIG. 8 is a block diagram of a system for identifying patterns and associated functions or structural features in molecules according to the present disclosure;

FIG. 9 is a block diagram for a system using a neuromorphic computing unit (NCU) for identifying radio frequency electromagnetic (RFE) signatures of a target molecule with associated functions or structural features, and application of an electromagnetic field according to a selected RFE signature to a substance according to the present disclosure; and

FIG. 10 is another diagram of an arrangement of MR sensor devices around a sample container according to the present disclosure.

### DETAILED DESCRIPTION

Ultra-low radio frequency energy therapy is based on measurement of the unique electrostatic potential of a target molecule. Every molecule has a unique electrostatic surface potential. This potential influences how a molecule interacts with other substances, such as proteins and other biological agents. Electron and charge transfer are central to many biological processes and are a direct result of interacting surface potentials. Applied artificial magnetic fields are capable of triggering a similar receptor-ligand response and conformational change in the absence of a physical substance, such as a molecular agonist or drug.

Application of unique and specific ultra-low radio frequency energy may induce electron and charge transfer in a defined bioactive target, altering cell dynamics to produce a therapeutic response. In at least some embodiments, to provide therapy, an ultra-low radio frequency energy cognate of a target molecule is delivered locally and non-systemically via a medical device. Pre-clinical and clinical studies suggest that ultra-low radio frequency energy therapy provides the ability to specifically regulate metabolic pathways and replicate known mechanisms of action for proven commercial drugs.

The present disclosure addresses a need for an integrated system that extracts frequency‑energy features in micro‑ or nano‑second timeframes; operates at sub‑watt power; and adapts an excitation signal waveform on the same timescale, thereby enabling real‑time interrogation and modulation of biological pathways.

In at least one aspect, the present disclosure describes a method for identifying patterns and associated functions or structural features in molecules, wherein the method includes measuring an electromagnetic field of a target molecule in at least two dimensions using an array of magnetic field sensor devices. At least one pattern in the measured electromagnetic field(s) of the target molecule is identified and associated with at least one function or structural feature of the target molecule. This method and other features and systems are described in U.S. Pre-Grant Publication No. 2025/0020737, which is assigned to the assignee of the present disclosure and incorporated by reference herein.

In at least some embodiments, the measured electromagnetic field of the target molecule may be compared with measured electromagnetic fields of other molecules to identify at least one common pattern in the measured electromagnetic fields of the target molecule and the other molecules. The common pattern with at least one common function or structural feature is associated with the target molecule and the other molecules.

In at least some embodiments, the electromagnetic field of a target molecule is mapped in three dimensions. In at least some embodiments, mapping the electromagnetic field includes mapping an electromagnetic field of a target molecule in at least two different planes.

In at least some embodiments, the magnetic field sensor devices are magnetoresistive (MR) sensor devices, in some cases tunneling magnetoresistive (TMR) sensor devices. In at least some embodiments, the magnetic field sensor devices may be superconducting quantum interference devices (SQUIDs). In at least some embodiments, the magnetic field sensor devices may be optically pumped magnetometer (OPM) sensor devices.

In at least some embodiments, an array of magnetic field sensor devices includes a combination of any two or all three of: TMR sensor devices, SQUIDs, and OPM sensor devices.

In at least some embodiments, identifying at least one pattern in the measured electromagnetic fields of the target molecule and other molecules includes identifying at least one statistical pattern in the measured electromagnetic fields. In at least some embodiments, the associating includes associating the at least one statistical pattern with a sequence, structural feature, or function of the target molecule.

In at least some embodiments, measuring the electromagnetic field of the target molecule includes solvating the target molecule in a solvent, placing the solvated target molecule in a sensor arrangement including the array of magnetic field sensor devices, subjecting the solvated target molecule to a stimulus, and acquiring a magnetic field generated by the solvated target molecule in response to the stimulus.

In at least another aspect, the present disclosure describes a system that includes a sensor array configured for measuring or mapping an electromagnetic field generated by a target molecule, a processing and storage arrangement configured for processing the electromagnetic field and storing the electromagnetic field after processing as a processed electromagnetic field, and a pattern recognition module configured for identifying one or more patterns in the processed electromagnetic field.

In at least some embodiments, the pattern recognition module is configured for identifying at least one statistical pattern in the processed electromagnetic field of the target molecule. In at least some embodiments, the pattern recognition module is further configured for associating the at least one statistical pattern to a sequence, structural feature, or function of the target molecule.

In at least some embodiments, the pattern recognition module is configured for comparing the processed electromagnetic field of the target molecule with measured electromagnetic fields of other molecules to identify at least one common pattern in the processed electromagnetic fields of the target molecule and the other molecules and associating the at least one common pattern with at least one common function or structural feature of the target molecule and the other molecules. In at least some embodiments, the system further includes at least one machine learning module for assisting the pattern recognition module to identify the at least one common pattern.

In at least some embodiments, the sensor array is configured for measuring or mapping the electromagnetic field of the target molecule in three dimensions. In at least some embodiments, the sensor array is configured for measuring or mapping the electromagnetic field of the target molecule in at least two different planes.

In at least some embodiments, the sensor array includes a plurality of magnetoresistive (MR) sensor devices, in some cases tunneling magnetoresistive (TMR) sensor devices. In at least some embodiments, the sensor array includes a plurality of superconducting quantum interference devices (SQUIDs). In at least some embodiments, the sensor array includes a plurality of optically pumped magnetometer (OPM) sensor devices. In at least some embodiments, the sensor array includes a combination of any two or all three of: TMR sensor devices, SQUIDs, and OPM sensor devices.

Examples of therapy delivery using ultra-low radio frequency energy can be found for example in U.S. Pat. Nos. 6,724,188; 6,952,652; 6,995,558; 7,081,747; 7,412,340; 10,046,172; 9,417,257; 11,103,721; 11,633,619; U.S. Pre-Grant Publications Nos. 2019/0143135 and 2019/0184188; and PCT Publication WO 2019/070911, all of which are incorporated herein by reference in their entireties. In at least some embodiments, for example, the delivery of ultra-low radio frequency energy includes the generation of a magnetic field having a field strength of up to 1 Gauss. In at least some cases, the delivery of ultra-low radio frequency energy includes the generation of a therapeutic electromagnetic signal having a frequency in a range, for example, of 0.1 Hz to 22 kHz or in the range of 1 Hz to 22 kHz.

Examples of systems affecting biologic activity with ultra-low radio frequency energy fields include experiments conducted to demonstrate the specificity and cellular effects of a specific ultra-low radio frequency energy targeting epidermal growth factor receptor, EGFR, on glioblastoma cell line U-87 MG. In such example, at 48 and 72 hours, EGFR inhibition by the ultra-low radio frequency energy reduced the level of EGFR protein by 27% and 73%, respectively. These data indicate that ultra-low radio frequency energy can inhibit gene expression at the transcriptional and protein levels, similar to what is observed with physical small interfering RNA (siRNA) inhibition. Specific EGFR knockdown effect was detected in U-87 MG cells treated with ultra-low radio frequency energy using an 80 gene PCR-based array. See, “Effects of Magnetic Fields on Biological Systems An Overview”; X. Figueroa, Y. Green, D. M. Murray, and M. Butters; EMulate Therapeutics; March 6, 2020.

In another example, ultra-low radio frequency energy therapy was provided as a cancer treatment for over 400 dogs with naturally occurring malignancies. Interim review of the first 200 dogs observed partial responses and complete responses in over 20 different tumor types. No clinically important or significant toxicities (Grade 3 or 4) were observed.

In some cases, conventional superconducting quantum interference devices (SQUID) have been used to measure the unique electrostatic potential of molecules. SQUIDs, however, can be bulky, expensive, and require cryogenic fluids for operation.

As described herein, a magnetoresistive (MR) sensor can be used in a single or multi-channel configuration to measure the magnetic field of a solvated target molecule and produce measurement signals. The measurement signals are processed and stored (for example, as a 24-bit WAV file) for uses such as, for example, therapy or drug discovery. In at least some embodiments, the bandwidth of the stored measurement signals is in a range from DC to 22 kHz or more. In at least some instances, particularly when using an MR sensor, the bandwidth is in a range of 0.1 Hz to 10 kHz.

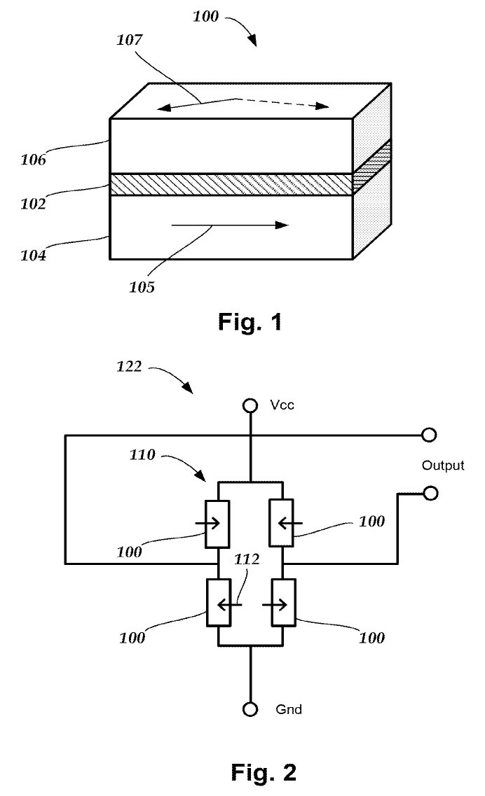

FIG. 1 illustrates one example of a magnetoresistive (MR) sensor 100 (in cases, also known as a tunnel magnetoresistive (TMR) sensor or magnetic tunnel junction (MTJ) sensor). The MR sensor 100 includes a thin film 102 of non-magnetic material between two ferromagnetic films that form a pin layer 104 and a free layer 106, respectively. The pin layer 104 has a direction of magnetization 105 that is pinned. Pinning can be accomplished by a variety of methods including forming the pin layer 104 of a material in a defined crystal structure. The direction of magnetization 107 of the free layer 106 follows the direction of an external magnetic field. For example, the free layer 106 can be formed of a material in an amorphous (e.g., non-crystalline) structure. Examples of MR devices are found in, for example, European Patent Application No. EP 2614770, incorporated herein by reference in its entirety.

The electrical resistance of the MR sensor 100 varies (in at least some embodiments, proportionally) with a relative angle between the directions of magnetization in the pin layer 104 and the free layer 106. Thus, by observing the resistance of the magnetoresistive sensor 100, the direction of the external magnetic field can be determined.

One or more MR sensors 100 can be used to measure the magnetic field by coupling to a DC power source. In FIG. 2 for example, a MR sensor device 122 includes a Wheatstone bridge arrangement 110 of four MR sensors 100 (where the arrows 112 indicate the direction of magnetization of the pin layer 106). The MR sensor device 122 can be used for differential temperature compensation. Any suitable magnetoresistive sensor device may be used, including devices incorporating tunneling magnetoresistive (TMR) or magnetic tunnel junction (MTJ) structures.

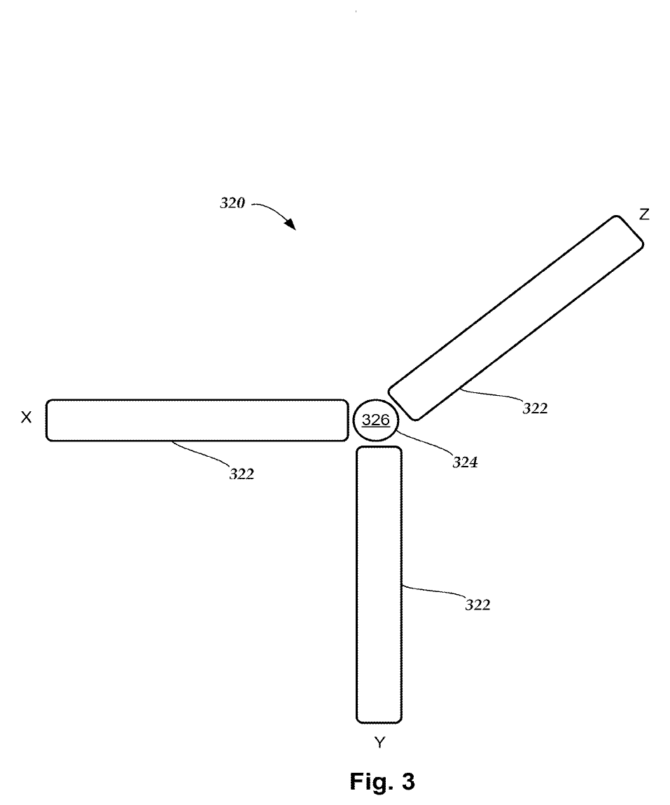

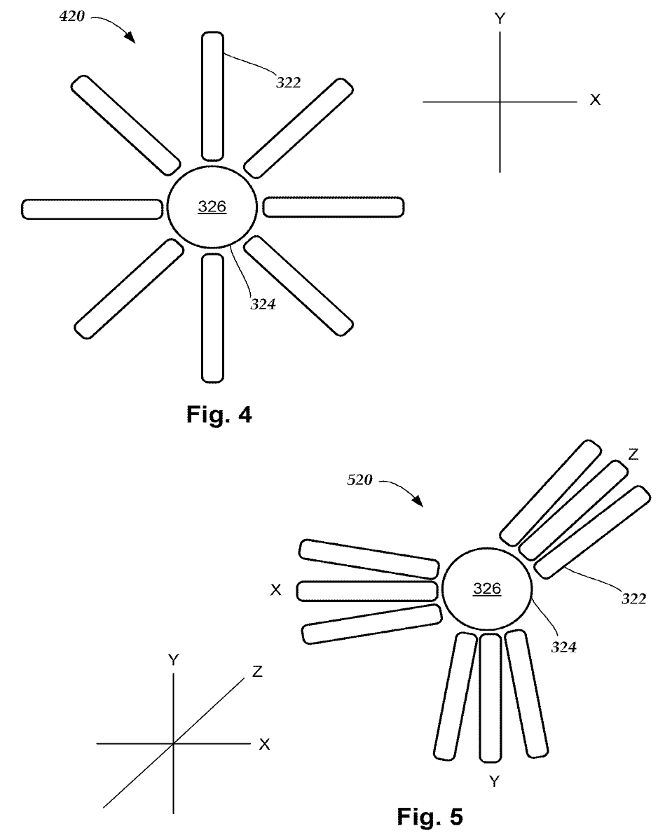

FIG. 3 illustrates a sensor arrangement 320 with multiple MR sensor devices 322 disposed around a container 324 with the target molecule 326 solvated in a solvent (for example, water, saline, phosphate buffered saline (PBS), plasma, or blood). The target molecule 326 can be any suitable target including, but not limited to, drug molecules (e.g., Taxol), oligonucleotides (e.g., RNA, mRNA, or the like), or any combination thereof.

In the illustrated embodiment, an MR sensor device 322 is positioned at the x, y, and z axes to measure the magnetic field arising from the electrostatic potential of the target molecule. Such measurement may include, for example, injecting noise into the sample in the container and recording the resulting magnetic field, as described in the references cited above. In at least some embodiments, the MR sensor device 322 can be a single MR sensor 100 or can be multiple MR sensors 100 arranged in the bridge illustrated in FIG. 2 or any other suitable arrangement.

FIG. 4 illustrates another sensor arrangement 420 with multiple MR sensor devices 322 disposed around the container 324 with the solvated target molecule. In the sensor arrangement 420, eight MR sensor devices 322 are arranged around in the container in the x-y plane.

FIG. 5 illustrates yet another sensor arrangement 520 with multiple MR sensor devices 322 disposed around the container 324 with the solvated target molecule. In the sensor arrangement 520, three MR sensor devices 322 are arranged around each of the x, y, and z axes. Other three-dimensional arrangements of MR sensor devices can be used including, for example, providing the arrangement 420 illustrated in FIG. 4 along multiple planes (for example, the x-y plane and the y-z plane).

The arrangements of MR sensor devices 322 illustrated in FIGS. 3-5 are examples of multi-channel configurations for recording the electrostatic potential of a target molecule. Single channel configurations with a single MR sensor device (or multiple MR sensor devices positioned together) can also be used.

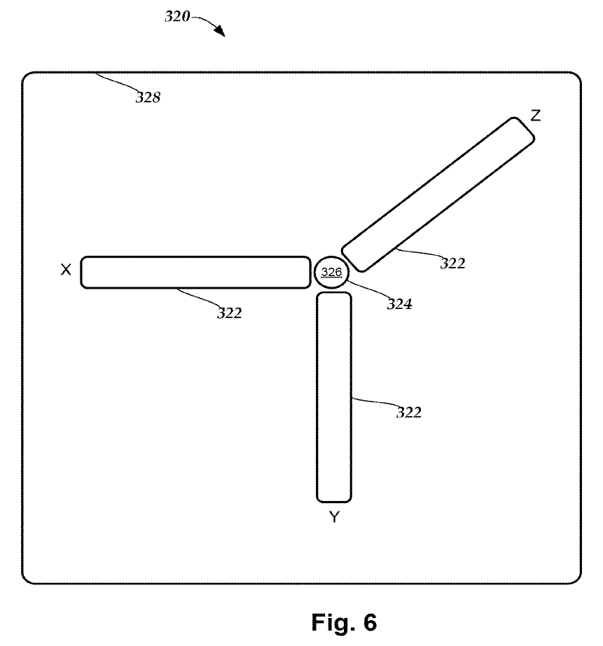

FIG. 6 illustrates the sensor arrangement 320 of FIG. 3 disposed within a shield 328 to reduce or remove the ambient magnetic field (such as the Earth's magnetic field) within the shield. The shield can be a passive shield (for example, made of mu-metal or other shielding material or a Faraday cage or the like) or an active shield (for example, one or more magnetic field generators to counter the ambient magnetic field) or any combination thereof.

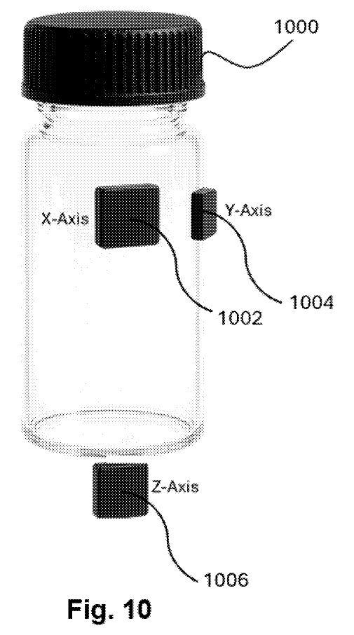

As a further example, FIG. 10 illustrates another arrangement of MR sensor devices 1002, 1004, and 1006 positioned with respect to a container 1000 according to the present disclosure. In this case, the container 1000 is a vial. The container 1000 is configured to hold a substance, such as a solvated target molecule or a biological sample as discussed herein. The three MR sensor devices 1002, 1004, and 1006 are arranged respectively along each of the x, y, and z axes to measure the magnetic field arising from the electrostatic potential of the substance in the container 1000.

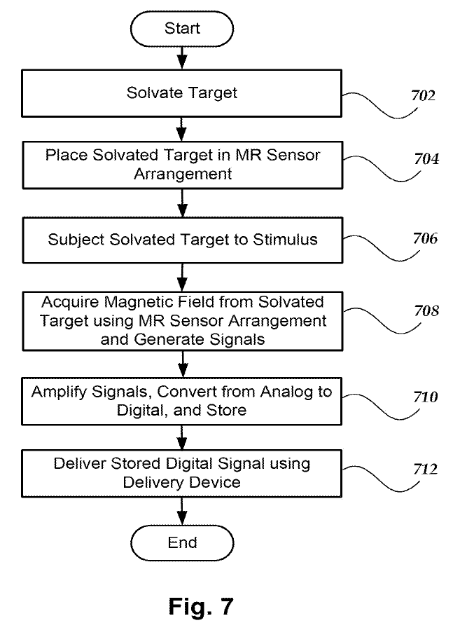

FIG. 7 depicts a workflow for generating and employing ultra-low radio frequency energy using the MR sensor devices described herein. In step 702, a target molecule is solvated in a solvent, such as, for example, water, saline, PBS, plasma, or blood. In step 704, the solvated target is placed in a MR sensor arrangement, such as one of the arrangements in FIGS. 3, 4, or 5, or any other multi-channel or single channel configuration or arrangement.

In step 706, the solvated target is subjected to a stimulus (for example, noise or other suitable signal) to elicit a response. In step 708, the MR sensor devices of the MR sensor arrangement acquire the magnetic field generated by the solvated target and the MR sensor devices generate output signals based on the acquired magnetic field. In step 710, the output signals from the MR sensor devices are amplified or otherwise processed, converted from analog to digital signals, and stored, e.g., in a digital memory.

In step 712, a stored digital signal may be selected and provided to a delivery device, such as a therapy delivery device, to deliver the one or more electromagnetic signals to a target to elicit a desired response based on the measurements obtained from the initial target molecule.

Electrostatic interactions of molecules are important to understanding the interaction of a drug with a biological system. Improvements in the determination of molecular force fields and improved visualization capabilities have enabled expansion beyond the ligand-only view of the electrostatics of drug interactions to include proteins, water, and ligands. Insights into the causes of ligand binding are now available to assist in drug discovery and design. Factors affecting molecular recognition of a drug include electrostatics, three-dimensional shape, and hydrophobicity. Creating accurate computational models for these factors for ligand and protein active sites can facilitate successful drug discovery and design.

Mapping of electromagnetic fields can also facilitate an understanding of how a biomolecule's ELF-EM (extremely low frequency electromagnetic) micro-amplifications play a role in ligand-receptor interactions. In at least some embodiments, “extremely low frequency” refers to frequencies in a range from 3 to 30 Hz with a corresponding wavelength in a range of 10,000 to 100,000 km. In at least some embodiments, a micro-amplification is a weak amplification, modification, or variation of the molecule's weak magnetic field.

Improving electromagnetic mapping can facilitate new therapeutic drug development. As described above, the electromagnetic field can be measured or mapped by injecting noise into a sample in a container and recording the resulting electromagnetic field using an array of magnetic field sensors such as, for example, MR sensors, SQUIDs, optically pumped magnetometers (OPMs), or the like or any combination thereof.

Biomolecular-drug interactions are formed when the valence electrons of complementary precursors (e.g., the biomolecule and drug) interact. In at least some instances, these interactions are initiated or maintained by electrostatic interactions including, but not limited to, Van der Waals forces, dipole-dipole interactions, ionic interactions, hydrogen bonds, or the like or any combination thereof.

As a chain of amino acids, a polypeptide expresses a unique electromagnetic field “fingerprint” that distinguishes it from other molecules. The specificity of a biomolecule's electromagnetic field also reflects the selectivity of its ligand-receptor interactions. Biomolecules are also affected by exogenous electromagnetic fields (EMFs). For example, in at least some instances, the crystalline structure of a microtubule—a ubiquitous cytoskeletal structure—can align with the cathode-anode orientation of an applied electromagnetic field.

All biomolecules (for example, proteins (polypeptides), RNA, DNA, or the like) can emit ELF-EM. Although the present invention is not limited to a particular theory, it is thought that the mechanism involves delocalized electrons along the biomolecule's backbone. A biomolecular ELF-EM “signature” can be driven by, for example, the nucleotide or amino acid sequence of the biomolecule (or any other arrangement of subunits of the biomolecule.) Each nucleotide or amino acid (or other subunit) contributes to the bulk electromagnetic field. For example, if the biomolecular structure is periodic or crystalline, the biomolecule is more likely to generate constructive interference between subunits (for example, repeating subunits.) Although the present disclosure is not limited to a particular theory, it is thought that these micro-amplifications of the molecule's ELF-EM field may at least partially drive intermolecular interactions, particularly those that are dependent on delocalized electrons.

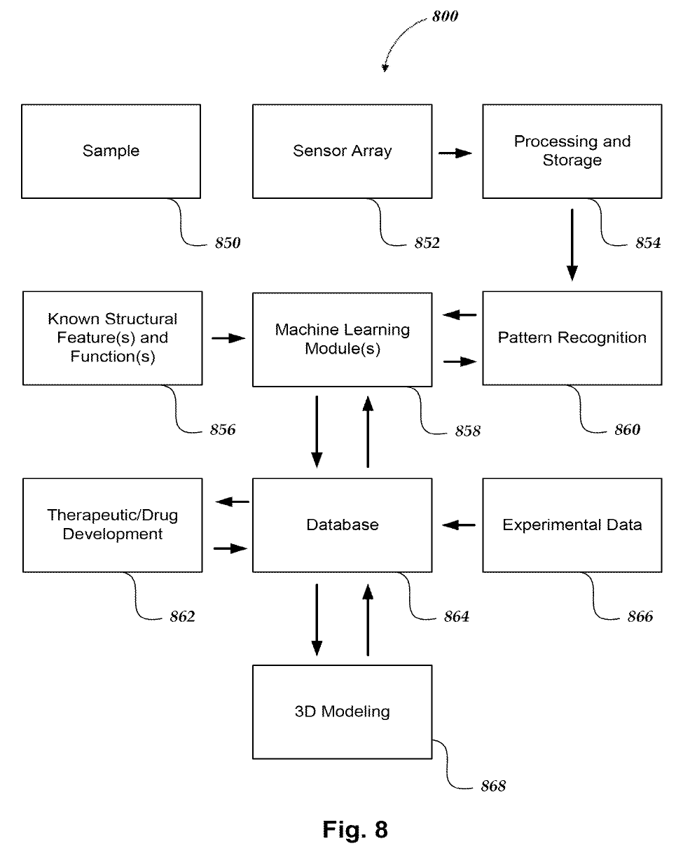

FIG. 8 is a block diagram of a system 800 for recognizing a pattern in a measured electromagnetic field of a sample compound which may be used, for example, for therapeutic or drug discovery or development. The system 800 includes a sensor array 852 for measuring or otherwise observing an electromagnetic field of a sample 850. The sensor array 852 can be an array of magnetoresistive (MR) sensors, SQUIDs, or OPM sensors, or the like or any combination thereof. The sensor array 852 can be a two-dimensional array, an array with sensors arranged in two or more planes, a three-dimensional array, or the like. The measured electromagnetic field is processed by one or more processors and stored in a database, e.g., in the processing and storage 854 in FIG. 8. The processors and database may be local to the sensor array 852 or remote from the sensor array.

A pattern recognition module 860 includes one or more pattern recognition or discovery algorithms, conducted according to software instructions, to identify statistical or other pattern(s) in the electromagnetic field measured by the sensor array 852. In at least some embodiments, the pattern recognition module may be conducted by the one or more processors in the processing and storage 854 utilizing one or more machine learning module(s) 858 and known structural feature(s) or function(s) of the sample 850. The identified pattern(s) of the sample 850 are associated with the sequence (where applicable), structural feature(s), and function(s) of the sample 850.

Suitable machine learning module(s) 858 can be used including, but not limited to, neural networks, decision trees, classifier algorithms, clustering algorithms, support vector machine algorithms, regression algorithms, nearest neighbor algorithms, or the like or any combination thereof. In at least some embodiments, the machine learning algorithm(s) 858 can be trained using data representing known molecules, known patterns, and known structural features and functions 856. In at least some embodiments, at least one threshold criterion (or any other suitable criterion) is used to determine when an identified pattern is associated with a particular structural feature or function or with similar identified patterns for other molecules (or the same molecule). In at least some embodiments, the system can include the threshold criteria (or any other suitable criteria) or a user can set or modify the threshold criteria (or any other suitable criteria).

In at least some embodiments, the pattern recognition module 860 or machine learning module(s) 858 may also include input from one or more people to facilitate the identification of patterns or association of a pattern to a function or structural feature.

In at least some embodiments, the association of a pattern with a function or structural feature of the sample 850 is stored in a database 864. In at least some embodiments, the pattern recognition module 860 or machine learning module(s) 858 learn from the identified patterns and develop governing rules for the patterns which may be stored in the database 864. The patterns and their association with function(s) or structural feature(s) or the governing rules for the patterns can be used for therapeutic or drug development or discovery 862 by facilitating designing of drugs or other therapeutics that will likely include desirable patterns.

In at least some embodiments, existing or new in vitro or in vivo data (for example, preclinical or clinical data) specific to one or more molecules can provide additional experimental data 866. The experimental data 866 can be stored in the database 864 and can be accessible to the machine learning module(s) 858. The machine learning module(s) 858 can access known structures, functions, patterns, 3D mappings of molecular magnetic fields, and the experimental data 866 to facilitate drug discovery and development or other applications.

In at least some embodiments, the measured electromagnetic field and, optionally, the identified patterns and associated structural features or functions can facilitate three-dimensional modeling 868 to create a model or map of the molecule or an electrostatic potential model or map of the molecule.

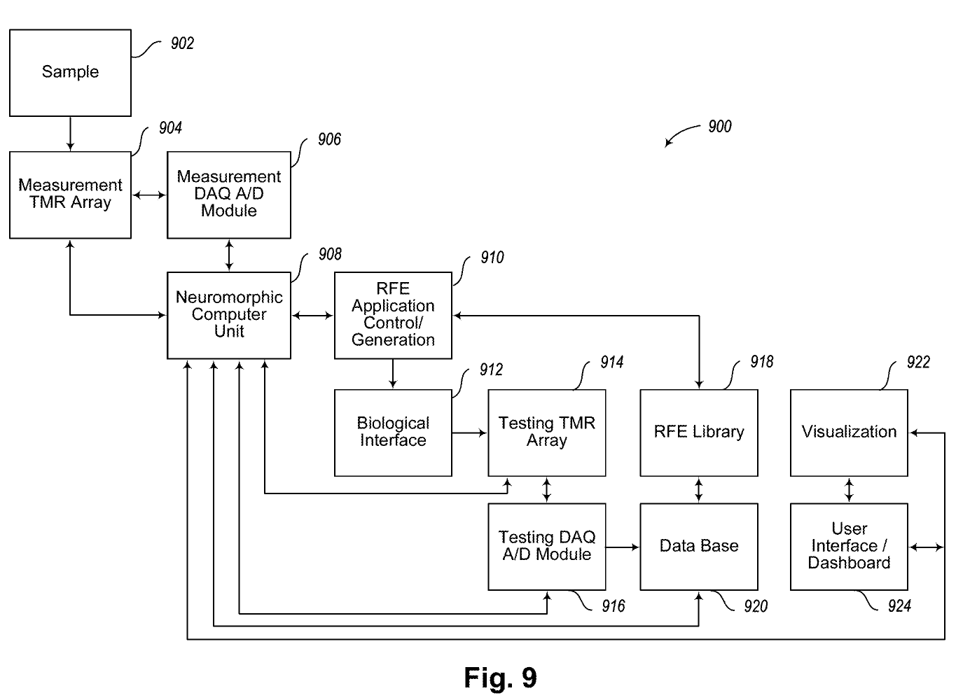

FIG. 9 is a schematic block diagram of a neuromorphic system 900 that enhances aspects of the present disclosure. The neuromorphic system 900 integrates neuromorphic computing to process radio frequency electromagnetic (RFE) molecular representations as described herein. By emulating the neural architecture and functioning of the human brain, the neuromorphic system 900 is able to adaptively process complex biological signals detected from a sample. It also enables non-invasive modulation of biological processes through RFE emissions to the sample, promoting real-time responsiveness in the sample with near-zero latency. This integration of neuromorphic computing creates an advantageous platform for biological research, allowing researchers to explore complex biological interactions of target molecules with biological samples with advantageous depth, timing, and precision.

The neuromorphic system 900 includes the neuromorphic computing unit (NCU) 908, which acts as a central processing hub for the system. The NCU 908 in this example utilizes neuromorphic processors, specifically spiking neural network (SNN) chips having spiking neural network cores, to mimic the behavior of biological neurons and synapses. These neuromorphic processors employ event-driven processing, reacting to spikes rather than continuous signals, enhancing efficiency and reducing power consumption, which is preferred for real-time applications.

The NCU 908 employs adaptive learning algorithms using machine learning models such as deep neural networks (DNNs), reinforcement learning, and generative adversarial networks (GANs). The neuromorphic system 900 continuously refines the machine learning models based on new data, learning from newly measured molecular RFE signatures that encode structural, functional, and energy states of target molecules. By identifying patterns in the RFE signals output from a sample, such as produced by active components within molecules, the NCU 908 can generate new RFE signatures using algorithms implemented by GANs and variational autoencoders (VAEs), for example. This capability allows the neuromorphic system 900 to create and, optionally, apply new RFE signatures to target molecules on biological interfaces with near-zero latency.

In this example the NCU 908 operates using SNNs inspired by biological neurons, employing event-driven processing. The NCU 908 refines machine learning models operating therein through continuous adaptive learning, such as using Hebbian plasticity, to adjust synaptic weights in response to real-time inputs. For example, when data from a biological sample indicates a positive therapeutic outcome after applying a specific RFE signal to the sample, synaptic weights within the models adjust immediately, strengthening associations that led to the positive outcome. Thus, over multiple iterations, the models in the NCU 908 continuously improve prediction accuracy without offline retraining.

In some cases, the NCU 908 is configured to generate new RFE signatures. The new RFE signatures can be applied to a sample by training a generative neural network (e.g., using GANs) to produce synthetic RFE signal patterns and use a discriminator network to evaluate the viability of the synthetic RFE signal patterns. VAEs are usable to compress the RFE signal patterns into latent representations and decode them into novel, plausible RFE signatures. Thus, the neuromorphic system 900 is able to systematically produce new candidate RFE signatures for testing.

In various implementations, an RFE signature includes measurable electromagnetic characteristics like frequency, amplitude, waveform shape, and phase information specific to a molecular target or therapeutic response. For example, an RFE signature for a particular protein interaction may include a waveform pattern with defined frequency peaks at 5 Hz and 18 kHz, specific amplitude modulations, and characteristic temporal phase shifts corresponding to known molecular interactions.

The neuromorphic system 900 includes an RFE molecular library 918 which functions as a digital repository storing the RFE signatures, essentially “digital twins,” of various molecules, including for example (without limitation) biological molecules such as siRNA, RNA, DNA, and proteins (e.g., therapeutic proteins), and small molecule compounds. In some embodiments, a digital twin is a digital twin of a drug molecule or a drug candidate molecule. The RFE molecular library 918 allows for quick retrieval of RFE signatures and application of RFE signals, with an update mechanism as described herein that enables the addition of new RFE signatures as RFE signal outputs by more molecules are measured and characterized by the neuromorphic system 900. The library 918 provides the neuromorphic system 900 with access to a comprehensive set of molecular representations in the stored RFE signatures for experimentation.

The neuromorphic system 900 includes an RFE application control and generation module 910 which manages the delivery of RFE signals to biological samples held in a biological interface 912. The biological interface 912 may be any substrate or container configured to hold a substance, such as plate with one or more wells or a container such as a vial, e.g., as illustrated in FIG. 10. Using control system circuitry arranged therein, the RFE application control and generation module 910 schedules RFE signal emissions from emitters in or under control of the module 910 to the sample(s) in the biological interface 912 based on experimental protocols and adjusts parameters such as intensity, frequency, and duration of the RFE emissions. The RFE application control and generation module 910 interfaces with the NCU 908 to make real-time adjustments to the RFE emissions. The RFE module 910 and associated emitters are capable of emitting complex and precise RFE signal patterns to a sample in the biological interface 912, ensuring high signal fidelity to accurately apply the RFE emissions, e.g., according to an RFE signature representing a target molecule during experimentation and testing.

The NCU 908 is configured to monitor real-time biological response (RFE signals) of the sample as measured during and/or after RFE emission. When the measured biological response of the sample deviates from a predicted or desired therapeutic response or efficacy, the NCU is configured to adjust, preferably immediately, RFE signal emission parameters such as frequency, amplitude, and phase. For example, if a real-time cell viability assay of a biological substance held by the biological interface 912 as described herein shows decreased efficacy, the NCU 908 instantly adjusts one or more parameters of the applied RFE signal (e.g., frequency, amplitude, etc.) to attempt improved therapeutic outcomes. In implementations using frequency‑energy feature vectors as described herein, the RFE application control and generation module 910 (RFE signal generator) is configured to adjust the one or more parameters of the applied RFE signal (radio frequency excitation signal) to maintain an extracted frequency‑energy feature vector (e.g., as determined from RFE signals obtained from the sample in the biological interface 912) within a specified range of a threshold frequency‑energy feature vector.

The biological interface 912 serves as the biological environment where electromagnetic field interactions with sample cells occur, and may include, for example, multi-well plates that house multiple cell cultures. Preferably designed to be transparent to electromagnetic fields, these plates ensure consistent RFE signal exposure across the samples in the wells. Environmental control systems operating on the biological interface 912 maintain optimal conditions, such as precise temperature regulation and controlled CO₂ levels, for cell viability in the multiple wells, ensuring reliable and reproducible results. Similar arrangements may be made with a sample container, such as the vial 1000 shown in FIG. 10.

Processed and newly-generated RFE signatures are stored in the RFE molecular library 918 with relevant metadata. The NCU 908 is configured to retrieve selected or newly-generated RFE signatures and determine emission parameters such as intensity, frequency range, modulation patterns, and duration, for RFE signals to be applied by the RFE application control and generation module 910 to the biological interface 912. Parameters are customized based on previous feedback (observed RFE outcomes) or specific experimental goals. The RFE application control and generation module 910 is responsible for converting digital RFE data into analog RFE signals suitable for emission to the biological interface 912. Signal amplification and modulation ensure that the RFE emissions satisfy the desired signal characteristics. RFE emitters in the RFE application control and generation module 910 are activated, emitting the desired RFE signal toward the sample(s) in the biological interface 912, with precise control over timing and signal characteristics as per the experimental design.

A tunnel magnetoresistance (TMR) sensor array 914 used for testing detects real-time changes in cellular activity in the sample(s) by measuring molecular electromagnetic fields of the sample(s). High-resolution TMR sensors are placed beneath or around cell cultures, for example, capturing three-dimensional RFE data generated by cellular processes as described earlier herein. A data acquisition system 916 collects sensor data from the TMR sensor array 914, preferably continuously, for analysis by the NCU 908.

A real-time data analysis and feedback control loop, including data visualization (e.g., display) 922 and user interface 924, provides a mechanism to fine-tune RFE emissions that are applied to the sample in the biological interface 912 based on cellular responses of the samples as measured by the testing TMR sensor array 914 and test DAQ module 916. The feedback control loop communicates analysis results (i.e., detected RFE signatures and processed data) from the NCU 908, enabling adjustment of RFE signal parameters in real time, such as modifying the RFE signal intensity, frequency, and/or phase. The data visualization interface 922 displays data and analysis results, preferably in real time, for researchers or other users, offering insights through (for example) interactive graphs, heatmaps, and three-dimensional models. Such visualization elements facilitate informed decision-making about representative biological interactions occurring in the sample in the biological interface 912.

The following workflow describes one example of using the neuromorphic system 900. Researchers may begin by selecting a molecule of interest, such as a drug, protein, or nucleic acid, and dissolving the molecule of interest in an appropriate solvent. The solvated molecular sample 902 is placed into a specialized measurement chamber or plate designed for optimal interaction with a measurement TMR sensor array 904 or other measurement sensor array including magnetic field sensors configured as described herein.

The measurement TMR sensor array 904 is configured and calibrated to detect RFE frequency ranges of interest produced by the sample 902. Sensors are positioned around the molecular sample chamber, e.g., as described earlier herein, and detect electromagnetic emissions from the solvated molecules, capturing analog electromagnetic signals, and generating molecular RFE signatures using the measurement data acquisition (DAQ) module 906. The RFE signatures result from molecular charges and interactions as sensed by the measurement TMR sensor array 904. The RFE signatures represent structural and/or functional aspects of the solvated molecules in the sample 902.

As noted, analog RFE signals detected by the TMR sensor array 904 are provided to the measurement DAQ module 906. High speed analog-to-digital converters (ADCs) in the DAQ module 906 convert the detected RFE signals into high-resolution digital data. Preferably, time stamps are attached as metadata to the digital data for synchronization and future reference, if needed.

Cell cultures, for example, are prepared and maintained under optimal conditions in the biological interface 912. Exposure to RFE signals according to a RFE signature is carefully controlled by the RFE application control and generation module 910 to ensure consistent exposure across the samples in the biological interface 912. The TMR sensor array 914 detects changes in cellular activity resulting from exposure to the RFE signals, capturing electromagnetic emissions from the samples indicative of physiological responses such as changes in gene expression and other cellular processes.

The digital data from the measurement DAQ module 906 is stored in a database in memory, e.g., database 920, typically without alteration. Preferably, data such as sample ID, solvent used, and measurement time and conditions are also recorded.

The NCU 908 is configured to receive the digital data from the measurement DAQ module 906 and, where appropriate, apply filtering algorithms to the digital data, e.g., to eliminate background noise. The NCU 908 uses mathematical models to identify key features of the RFE signatures, such as frequency peaks and amplitude variations in the detected RFE signals. In some cases, the NCU 908 is configured to extract a frequency‑energy feature vector from the RFE signal detected by the plurality of magnetic field sensors in the testing TMR sensor array 914. Neuromorphic algorithms operating in the NCU 908 enable the NCU to recognize patterns in the RFE data and classify the molecular RFE signatures, for example comparing newly-generated RFE signatures with RFE signatures stored in the RFE molecular library 918.

The NCU 908 is configured to employ mathematical models to extract and analyze electromagnetic features (e.g., frequency peaks, temporal patterns, and the like). Neuromorphic processing provided by the NCU 908 uniquely uses spike-based, event-driven detection, enabling immediate identification of these features at hardware level, significantly reducing computational delay compared to traditional architectures.

Neuromorphic algorithms operate by converting electromagnetic (RFE) data into spike-based events. Each spike encodes RFE data such as amplitude and frequency. The NCU 908 may then classify these RFE data (RFE signatures) through real-time pattern recognition. For example, when a new RFE signal pattern is received, “neurons” in the NCU 908 representing known therapeutic signatures fire spikes simultaneously, immediately classifying and matching the pattern to known therapeutic effects without lengthy computational steps.

Analog biological response signals from the samples in the biological interface 912 are amplified, filtered, and converted into digital data by the testing DAQ module 916, preferably with timestamps attached. The digital biological feedback data is securely stored in the database 920, including metadata such as cell type and experimental conditions, for example. The NCU 908 processes this digital biological feedback data, filtering and normalizing it to identify correlations between RFE signal emissions from the module 910 and biological responses in the samples as detected by the testing TMR sensor array 914. The NCU 908 can assess the effectiveness of the RFE signal emissions in eliciting desired responses in the samples and adjust internal models for improved predictions and response by adjusting the RFE signal emissions.

An RFE signal generator such as the RFE application control and generation module 910 is configured to synthesize a radio frequency excitation signal responsive to the RFE signal data, which may be arranged in form of a frequency‑energy feature vector. An emitter assembly such as in the RFE application control and generation module 910 is configured to deliver the radio frequency excitation signal to the sample (target) in the biological interface 912. In this manner, the RFE application control and generation module 910 is coupled to the magnetic field sensors in the testing TMR array 914 and provide the RFE signal generator that is operable to adjust the radio frequency excitation signal. In various implementations, the adjustment is made to reduce a difference between the frequency‑energy feature vector extracted from the RFE signal received from the sample and a desired or intended frequency‑energy feature vector, which may be a threshold frequency‑energy feature vector. The threshold frequency‑energy feature vector may be representative of a structural RFE motif that is linked to a desired therapeutic effect in the sample. This adaptive learning incorporates findings from the testing of RFE signals in real time to refine the application of future RFE emissions to the biological interface 912.

The NCU 908 is configured to assess effectiveness by comparing real-time measured biological responses to expected, anticipated, or desired outcomes. In some cases, the NCU 908 is configured to correlate a biological response of the sample with the extracted frequency-energy feature vector and arrange the threshold feature-energy vector to represent a desired biological response of the sample. Differences between the measured biological responses and the expected, anticipated, or desired biological responses (outcomes) trigger immediate updates in the internal predictive model, refining future RFE emissions. For example, if an RFE signal emission expected to inhibit a particular gene expression fails to achieve the expected inhibition in a cell assay (samples in the biological interface 912), the NCU 908 modifies the RFE signal parameters in real-time to test a new frequency or waveform and update predictive models accordingly.

Researchers may monitor real-time data and system status via the visualization interface 922, which is configured to display data on RFE signal emissions, detected biological responses, and system adjustments being made. Interactive graphs and alerts allow for immediate interpretation and decision-making. Researchers can adjust settings, introduce new variables, or alter protocols in real time through the user interface 924. Changes made are executed by the NCU 908, preferably immediately, ensuring that the user input has a near-instant effect on the testing.

Experimental data, including raw signals, processed information, settings, and system logs, are preferably securely stored in memory, e.g., in the database 920. This ensures traceability and supports compliance reporting. Data in the database 920 is readily available for in-depth analysis, reporting, or auditing.

The ability of the NCU 908 to analyze RFE signatures and identify electromagnetic patterns associated with biological activity accelerates the screening of vast chemical libraries, e.g., to identify promising drug candidates.

Recurring structural RFE motifs are repetitive electromagnetic waveform patterns consistently observed to correlate with, e.g., particular therapeutic effects. For example, a specific sequence of frequency shifts and amplitude modulations of an applied RFE signal repeatedly observed to reduce inflammation could be identified as a therapeutic motif.

By recognizing recurring structural RFE motifs linked to desired therapeutic effects, the neuromorphic system 900 facilitates detailed structure-activity relationship (SAR) analyses, rapidly correlating structural features of molecules under measurement with biological functions. This guides medicinal chemists and researchers in discovering compounds optimized for potency, selectivity, and safety. The capability to identify and generate new RFE signatures allows the NCU 908 to simulate novel compounds by generating respective RFE emissions and predicting their interactions with biological samples, bridging the gap between computational predictions and experimental validation. This can greatly reduce the time and cost of drug development.

Researchers are thus able to receive data from the NCU 908 indicating electromagnetic signatures correlated with therapeutic efficacy, selectivity for targeted receptors, and minimal off-target effects. For example, the NCU 908 may be configured to identify an RFE signature linked to selective receptor activation without triggering adverse pathways. Medicinal chemists may use this insight to synthesize or modify molecular structures predicted to produce similar RFE signatures, guiding optimization efforts.

Furthermore, by testing the effect of RFE signals on patient-derived cells according to RFE signatures, the NCU 908 and RFE application control and generation module 910 can assess individual responses of the patient-derived cells to various drugs or treatments (as represented by the applied RFE signals), aiding in the development of personalized medicines and precision therapeutic strategies. Realtime feedback from the biological interface 912 via the testing TMR sensor array 914 and testing DAQ module 916 allows the NCU 908 to dynamically adjust therapeutic interventions by modifying the RFE signatures being applied to the biological interface 912 or by suggesting alterations in treatment protocols based on the immediate cellular responses of the patient-derived cells. This enables customization of therapies to a particular patient to enhance efficacy and minimize adverse effects.

Learning from RFE signatures that encode (or represent) target molecular structures and energy states helps uncover how specific conformations influence biological function. The NCU 908 is configured to model biological interactions at the atomic level in a matter agnostic to particular molecules and compounds. The NCU 908 is thus able to reveal underlying biological mechanisms, e.g., of enzyme catalysis, receptor activation, and signal transduction. Analyzing energy states involves examining RFE signals related to molecular conformations and their associated energies. By analyzing energy states detected by the testing TMR sensor array 914, the NCU 908 provides insights into molecular dynamics, such as folding pathways and stability, which can be crucial for designing stable biopharmaceuticals and understanding disease-related misfolding processes. Insights gained may include stability, conformational flexibility, and binding affinity information. These insights inform researchers about molecular interactions, guiding structural modifications and therapeutic strategy decisions.

The NCU 908 integrates new testing data from the testing DAQ module 916 into machine learning algorithms operating in the NCU, preferably continuously, enabling dynamic adjustment of processing parameters and RFE signal emissions by the RFE application and control module 910. The iterative process refines the performance of the neuromorphic system 900 with each cycle, enhancing accuracy and efficacy over time. Key metrics are tracked by the NCU 908 to assess improvements and guide future experimentation.

The ability to instantaneously measure, generate, and test new RFE signatures facilitates high-throughput experimentation. Researchers can iteratively modify molecular structures as represented by respective RFE signatures that are applied to a sample in the biological interface 912, and immediately observe biological outcomes, significantly accelerating discovery cycles. The adaptive learning of the neuromorphic system 900 allows new hypotheses to be quickly identified based on emerging RFE patterns observed in the RFE testing.

In various implementations, the NCU 908 is a purpose‑built, wafer‑scale slice of neuromorphic silicon engineered to treat ultra‑low‑frequency electromagnetic biosignals as first‑class spiking events. In one example, 9 mm × 9 mm die, fabricated in 65 nm CMOS, is configured to house four neuro‑tiles, each incorporating 256 leaky‑integrate‑and‑fire (LIF) neurons, a dual‑port sensor first-in first-out (FIFO), and a 4 kB reference‑fingerprint look-up table (LUT). The FIFO is arranged to isolate a 10 kS s⁻¹ ADC clock domain from the synchronous neuron fabric, while the LUT assigns a 2‑bit role flag, for logic, sense, or actuate roles, to every frequency, phase, and amplitude (f, Δf, A) triplet.

Signal input circuitry may include a hard‑wired peak‑finder array consisting of a number of slope comparators (e.g., 256) clocked at, for example, 40 MHz, and a 1‑tap FIR differentiator configured to flag zero‑crossings that exceed a programmable threshold θ. Successive peaks captured in a defined period (e.g., 60 ns) are passed to a triplet builder that updates a 24‑bit frequency counter, an 11‑bit log₂ frequency‑difference register, and a 10‑bit amplitude quantizer. In a single additional cycle, a hashed index may be configured to address the on‑core LUT, yielding a role flag that is concatenated to a 9‑bit triplet identifier. The resulting 11‑bit spike token is broadcast over an address‑event representation (AER) bus with a 25 ns handshake.

Because, in this example, computation is strictly event‑driven, dynamic power scales with biosignal activity rather than with ADC sample rate. For example, in a quiescent cardiomyocyte preparation, the average neuron fires 0.12 Hz, resulting in a silicon utilization < 5 % and an active power budget below 100 mW. End‑to‑end latency, from analog peak to classified spike, is measurable at approximately 100 ns, which is a three order of magnitude improvement over a 4 k‑point graphic processing unit (GPU) Fast Fourier Transform (FFT) requiring approximately 88 μs including host transfer. Energy per spectral decomposition is on the order of 1 nJ versus 0.9 μJ on a GPU, translating to a 900‑fold efficiency gain.

In other words, a conventional 4 k‑point radix‑2 FFT performs 48 k complex multiplications per update. In contrast, the spike‑FFT implemented in the NCU 908 relies on 64 frequency‑tuned LIF neurons whose membrane potentials integrate only coincident spikes. Idle neurons leak at 25 fJ per 100 μs, and active spiking costs 40 fJ per event, yielding approximately 1 nJ per complete spectral update, as noted being three orders of magnitude below a clock‑driven digital signal processor (DSP). A proportional–integral–derivative (PID) circuit in the NCU 908 may compare live Fourier spectra from the TMR sensor array with the target or desired feature vector and alter the RFE emitter phase/amplitude every 20 ms, for example. In this manner, a –1 dB drift at 7 Hz was countered by boosting the signal emitter amplitude by 3 %, restoring a lock on the target or desired feature vector within 180 ms.

A neuromorphic radio frequency electromagnetic system as described herein may thus include a plurality of magnetic field sensors arranged to detect an RFE signal from a target (e.g., sample), and an NCU configured to extract a frequency‑energy feature vector from the RFE signal detected by the magnetic field sensors and to generate a new RFE signal based on an observed RFE outcome or an experimental objective. The system may further include an RFE molecular library communicatively coupled to the NCU, the NCU being configured to retrieve one or more RFE signals from the RFE molecular library to generate a new RFE signal based on an observed RFE outcome or an experimental objective. The system may further include a waveform generator configured to synthesize a radio frequency excitation signal responsive to the frequency‑energy feature vector, and in some cases the NCU is configured to computationally generate a molecular candidate having an RFE signature corresponding to the new RFE signal, wherein the new RFE signal encodes a predicted structural or functional molecular attribute of the molecular candidate.

As can be seen, the neuromorphic system 900 provides real-time measurement, generation, and testing of RFE signatures. Neuromorphic computing by the NCU 908 significantly enhances this capability by providing real-time spike-based data processing and adaptive learning at microsecond or nanosecond latencies, eliminating the delays typical of traditional computational systems.

For example, when conducting research on natural products, the NCU 908 can identify active constituents within complex natural extracts by recognizing patterns in the measured RFE signals and link such RFE patterns to bioactivity of the target substance (e.g., natural extract) being measured. This can greatly streamline the identification and isolation of therapeutic agents from natural sources, such as plants, marine organisms, and microbial cultures, for example. Also, by identifying RFE signal patterns associated with toxicity or undesirable interactions, the NCU 908 also helps preemptively eliminate future application of harmful compounds. This enhances safety profiles by guiding the design of target molecules with reduced off-target effects.

Detecting and generating the RFE signatures of biomolecules involved in metabolic pathways enables the NCU 908 to identify and suggest modifications for enhanced efficiency or treatment. The neuromorphic system 900 supports the engineering of microbes or cells to produce valuable compounds, such as biofuels or pharmaceuticals. The neuromorphic system 900 assists in designing proteins or nucleic acids with specific functions, stability, or interactions, contributing to the development of novel enzymes, therapeutic antibodies, or gene-editing tools.

The NCU 908 is configured to identify RFE signatures correlated with efficient metabolic processes, such as optimal enzyme activity. The NCU 908 generates and tests variations of these RFE signatures, in effect suggesting molecular structural modifications that would replicate or enhance these efficient processes. For example, an RFE signature correlated with enhanced glucose metabolism by a key enzyme may be identified. The NCU 908 then suggests a slightly modified molecular structure by way of an adjusted RFE signature that is found to further optimize enzyme efficiency, guiding medicinal chemists to test this improved molecule experimentally.

By applying RFE signals to a biological sample according to an adjusted RFE signature, the NCU 908 can, for example, influence stem cell fate and promote differentiation into specific cell types. This may be used to enhance the production of cells needed, e.g., for tissue repair or replacement therapies. By understanding how cells respond to different materials at the molecular level, as represented by respective RFE signatures, the neuromorphic system 900 aids in designing scaffolds that promote tissue integration and healing.

Furthermore, early detection of structural features associated with toxicity allows for modifications before clinical testing, enhancing patient safety and reducing the likelihood of late-stage drug failures. Real-time testing of RFE signals on biological interfaces, such as biological interface 912, helps in formulating drugs with optimal release profiles and bioavailability.

The neuromorphic system 900 also facilitates the study of intricate mechanisms like protein-protein interactions, signaling cascades, and gene regulation. It enables researchers to manipulate and observe biological systems with unprecedented precision, promoting cross-field collaborations and driving holistic scientific advancements.

In at least one implementation, the neuromorphic system 900 may apply an RFE signal according to an RFE signature of identified siRNA. By utilizing siRNA RFE signatures to modulate specific molecular action, the neuromorphic system 900 offers a novel approach to non-invasively control cellular behavior. This has significant potential in therapeutics, enabling the treatment of diseases by correcting bioelectric aspects of target biology. The system may create and apply siRNA RFE signatures targeting ion channels for example, adjusting RE emissions in real time based on detected cellular responses, exemplifying its innovative potential in cellular manipulation.

The system's capabilities have profound implications across multiple domains, including regenerative medicine, cancer research, neuroscience, synthetic biology, and environmental monitoring. It accelerates the pace of discovery, deepens our understanding of biological processes, personalizes healthcare, and fosters innovation across these fields.

As described above, significant advantages are achieved using the neuromorphic computing (NC) described herein. The NCU 908 can achieve instantaneous data analysis by emulating the brain's event-driven, massively parallel neural network architecture. Unlike traditional computing architectures (CPUs and GPUs), NC processes data through spiking neural networks, offering significant advantages in terms of speed, energy efficiency, and real-time responsiveness.

Instantaneous (or near instantaneous) achieves biological mimicry. Neuromorphic systems use "spiking neurons," modeled closely after biological neurons. These neurons process and transmit information as discrete spikes or pulses, called action potentials. This enables sparse computation, in which computation only occurs when neurons spike, dramatically reducing the number of computations. In contrast, traditional systems constantly perform computations regardless of whether new meaningful data is available. Minimal latency is achieved. Neurons fire instantaneously in response to stimuli. Each spike encodes timing information precisely, allowing real-time event processing. Additionally, a massively parallel architecture is achieved using parallel neurons. Neuromorphic chips contain thousands to millions of independent neurons working concurrently. Each neuron can simultaneously analyze different portions of incoming data. It also utilized decentralized memory. Unlike traditional computing (where memory access is a bottleneck), neuromorphic systems use distributed synaptic memory. Each neuron typically has local, dedicated memory, minimizing data transmission and speeding response times.

Additional advantages include localized, low-overhead communication. Neuromorphic systems use address-event representation (AER) protocols for communication. Each spike event is transmitted individually with a time-stamp, drastically reducing overhead. No bulky data buffers or frame-based buffering are needed. Rapid handshake protocol is viable. For instance, neuromorphic systems commonly implement event transmission handshakes as fast as 25 ns, achieving real-time spike broadcasting with extremely low latency.

As for hardware-level spike detection and feature extraction, various implementations may utilize hardware peak-finders, more specifically specialized circuitry (e.g., slope comparators, differentiators) that instantaneously detect peaks or changes in sensory signals. The hardware then immediately encodes these peaks as spikes. Dedicated circuitry instantly builds feature representations—such as frequency (f), frequency change (Δf), and amplitude (A)—from these detected peaks, providing immediate contextualized data. Role-based classification includes instantaneous hash lookups via small on-chip lookup tables (LUT) that classify these spike features as logic, sense, or actuate events immediately upon detection.

As a result, real-time adaptive learning is achieved, for example using Hebbian plasticity. Neuromorphic neurons use Hebbian learning, updating connections in real-time as data arrives, instantly adapting to new patterns and stimuli without needing large training batches. Neuromorphic systems implement real-time reinforcement learning. As soon as a new data event (spike) occurs, weights and parameters are updated instantaneously, allowing the model to continuously refine its predictions and adapt dynamically.

Great power and energy efficiency is obtained. Neuromorphic chips typically consume fractions of the power used by GPUs or CPUs performing similar analysis. This efficiency arises because neurons consume energy mainly during spike transmission (around tens of femtojoules per spike), while idle neurons leak minimal current. Thus, neuromorphic systems can handle continuous, high-throughput data streams instantaneously, with minimal power consumption.

As for sensor arrays (TMR, SQUID, OPM), neuromorphic systems immediately interpret magnetic or electromagnetic field data, instantly converting raw analog inputs to categorized spikes, instantly triggering decision-making or control responses. By way of example, implementations of a neuromorphic platform may immediately convert biosignals (e.g., Ca²⁺ transient spikes) into instantaneous spikes, updating computational models, PID control loops, and generating immediate adaptive waveform outputs—all within sub-microsecond latency. This approach enables instantaneous adaptive responses in fields like medical prosthetics, sensory-rich robotics, or real-time scientific experimentation.

The various embodiments described above can be combined to provide further embodiments. All of the patents, applications, and publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. A neuromorphic computing system for molecular electromagnetic signature detection and adaptive radio frequency electromagnetic (RFE) modulation, comprising:
a radio frequency electromagnetic (RFE) application control and generation module configured to apply an RFE signal to a sample in a biological interface according to characteristics of an RFE signature;
a magnetoresistive (MR) sensor array arranged with respect to the biological interface to detect RFE signals from the sample;
a testing data acquisition (DAQ) module communicatively coupled to the MR sensor array to generate RFE signal data based on the detected RFE signals; and
a neuromorphic computing unit communicatively coupled to the testing DAQ module to receive the RFE signal data,
wherein the neuromorphic computing unit is configured to adjust in real time the RFE signal applied to the sample in the biological interface.

2. The neuromorphic computing system of claim 1, wherein the RFE signal applied to the sample is generated according to the characteristics of an RFE signature selected from a library containing a plurality of RFE signatures.

3. The neuromorphic computing system of claim 2, wherein the RFE signatures are obtained from measurement of RFE signals of solvated molecules and are representative of molecular structure and/or function of the solvated molecules.

4. The neuromorphic computing system of claim 1, wherein the neuromorphic computing unit is configured to determine a difference between the detected RFE signals and an RFE signal indicative of desired therapeutic benefit, and adjust in real time the RFE signal applied to the sample in the biological interface to minimize the difference.

5. The neuromorphic computing system of claim 1, wherein the magnetoresistive (MR) sensor array is comprised of tunnel magnetoresistance (TMR) sensors arranged to detect real-time changes in biological activity in the sample by measuring the RFE signals from the sample.

6. The neuromorphic computing system of claim 1, wherein the neuromorphic computing unit is configured to recognize one or more patterns in the RFE signal data and generate an RFE signature representative of the sample based on a pattern in the RFE signal data.

7. The neuromorphic computing system of claim 6, wherein the neuromorphic computing unit is configured to compare the generated RFE signature with one or more RFE signatures stored in a library containing a plurality of RFE signatures.

8. The neuromorphic computing system of claim 6, wherein the neuromorphic computing unit uses spike-based, event-driven processes to recognize the one or more patterns in the RFE signal data.

9. The neuromorphic computing system of claim 1, wherein the neuromorphic computing unit is configured to correlate the RFE signal applied to the sample by the RFE application control and generation module with a biological response observed the detected RFE signals and assess an effectiveness of the RFE signal in eliciting a desired biological response in the sample.

10. The neuromorphic computing system of claim 9, wherein the effectiveness is assessed by comparing a real-time measured biological response represented in the detected RFE signal to an expected, anticipated, or desired biological response, and a difference between the measured biological response and the expected, anticipated, or desired response triggers the real-time adjustment in the RFE signal applied to the sample.

11. A closed‑loop radio‑frequency excitation system, comprising:
a plurality of magnetic field sensors arranged to detect a radio frequency electromagnetic (RFE) signal from a sample;
a neuromorphic computing unit (NCU) configured to extract a frequency‑energy feature vector from the RFE signal detected by the plurality of magnetic field sensors;
an RFE signal generator configured to synthesize a radio frequency excitation signal responsive to the frequency‑energy feature vector;
an emitter assembly configured to deliver the radio frequency excitation signal to the target; and
a radio frequency electromagnetic (RFE) application control and generation module coupled to the magnetic field sensors and the RFE signal generator and operable to adjust the radio frequency excitation signal to reduce a difference between the extracted frequency‑energy feature vector and a threshold frequency‑energy feature vector.

12. The radio‑frequency excitation system of claim 11, wherein the NCU comprises a spiking neural network core.

13. The radio‑frequency excitation system of claim 11, wherein the RFE signal generator comprises a variational autoencoder coupled to a generative adversarial network.

14. The radio‑frequency excitation system of claim 11, whereinthe threshold frequency‑energy feature vector is representative of a structural RFE motif that is linked to a desired therapeutic effect in the sample.

15. The radio‑frequency excitation system of claim 11, whereinthe NCU is configured to correlate a biological response of the sample with the extracted frequency‑energy feature vector and arrange the threshold feature-energy vector to represent a desired biological response of the sample.

16. The radio‑frequency excitation system of claim 11, wherein the RFE signal generator is configured to adjust one or more parameters of the radio frequency excitation signal to maintain the extracted frequency‑energy feature vector within a specified range of the threshold frequency‑energy feature vector.

17. A neuromorphic radio frequency electromagnetic system, comprising:
a plurality of magnetic field sensors arranged to detect a radio frequency electromagnetic (RFE) signal from a target;
a neuromorphic computing unit (NCU) configured to extract a frequency‑energy feature vector from the RFE signal detected by the magnetic field sensors and to generate a new RFE signal based on an observed RFE outcome or an experimental objective.

18. The system of claim 17, further comprising an RFE molecular library communicatively coupled to the NCU, the NCU being configured to retrieve one or more RFE signals from the RFE molecular library to generate a new RFE signal based on an observed RFE outcome or an experimental objective.

19. The system of claim 17, further comprising a waveform generator configured to synthesize a radio frequency excitation signal responsive to the frequency‑energy feature vector.

20. The system of claim 17, wherein the NCU is configured to computationally generate a molecular candidate having an RFE signature corresponding to the new RFE signal, wherein the new RFE signal encodes a predicted structural or functional molecular attribute of the molecular candidate.
